(19) United States
(12) Patent Application Publication
(10) Pub. No.: US 2026/0096747 A1
(43) Pub. Date: Apr. 9, 2026

(54) Mobile Rheometer Device, System and Method for Investigating Rheological Properties of Human, Animal or Plant Components

(71) Applicant: NETZSCH-Gerätebau GmbH, Selb (DE)

(72) Inventors: Sabrina Küspert, Selb (DE); Florian Rummel, Selb (DE); Levente Szántó, Hof (DE); Rebekka Taubmann, Selb (DE)

(21) Appl. No.: 19/351,552

(22) Filed: Oct. 7, 2025

(30) Foreign Application Priority Data
Oct. 9, 2024 (DE) 10 2024 129 204.0

(51) Int. Cl. A61B 5/145; A61B 5/00; G01N 11/00; G01N 33/00

(52) U.S. Cl. CPC A61B 5/14503; A61B 5/4238; G01N 11/00; G01N 33/0098

(57) ABSTRACT

A mobile rheometric device for examining rheological properties of human, animal, or plant components. Such a mobile rheometric device includes a rheological measurement device and, coupled thereto, a computer-based functional unit with user program. The mobile rheometric device includes, in addition, a transport unit in which the rheological measurement device and the computer-based functional unit coupled thereto are positioned, wherein the transport unit is configured to be placed at least temporarily in an internal component of a human, animal, or plant body, so that the rheological measurement device can interact with the human, animal, or plant body when brought in contact.

### TECHNICAL FIELD

The present invention relates to a mobile rheometric device and a system as well as a method for examining rheological properties of human, animal, or plant components.

### BACKGROUND

To be able to generate far-reaching findings concerning human, animal, or plant components, for example in the form of individual samples, such components, or merely samples thereof, are frequently extracted from the organism and submitted to complex laboratory analysis. A common alternative is to analyze a fluid that is related to the tissue or organism that is to be studied.

Thus, for instance, samples are extracted from a human being during a biopsy or a surgical procedure by medical specialists and are then turned over to pathologists. In the pathology work, a histological investigation often follows, typically with the staining of tissue and subsequent pathological diagnosis. Tissue samples can also be prepared by tissue sectioning for purposes of further analysis of these individual sections.

Rather than a histological examination with staining and the use of microscopic devices, rheological techniques may also be used, such as in order to perform a medical diagnosis. These techniques can include, for example, rotation rheometers or capillary rheometers. In addition, falling-sphere viscometers or microfluidic devices can be employed. Pathological tissue is distinguished from the physiological condition, for example, by its viscous, viscoelastic, or elastic properties.

For example, stenoses or similar disorders can be investigated more closely by such means. Bodily fluids such as saliva, blood, and the like are likewise distinguished in their viscous, viscoelastic, or elastic properties when a modification occurs. Such a situation is known, for example, in association with sickle cell anemia. In such cases, knowledge of rheological characteristics of physiological or pathological properties can be put to diagnostic use.

In this process, measurements of one or more rheological values, such as (sheer) viscosity, extensional viscosity, shear stress, viscoelastic properties, storage modulus, or loss modulus can be taken. Such measurements can include absolute or relative values. A particular sample can typically be set into oscillation or vibration. Alternatively, rotation or motion in the form of a flow can also be foreseen. In addition, bodily determined circumstances of samples can already assume forms of oscillation, vibration, rotation, or flowing, and thus this circumstance can be useful for rheological methods.

Procedures of this kind employing various applications of such techniques can entail a certain time commitment. In addition, in transporting samples a certain logistical infrastructure is required in order to avoid impairments to samples which are often sensitive during this time. In this context, modifications of samples are likely to occur despite all precautions and cannot be entirely ruled out. Biological sample material is generally modified fairly rapidly, and thus subsequent investigations and/or analysis becomes difficult.

In the aforementioned cases of biopsy and extraction during surgery, the necessary medical infrastructure is often available only with maximal providers. Supply and diagnosis in remote locations, such as at high elevations or at sea, can accordingly prove difficult, since the necessary medical infrastructure is either only available with difficulty or not present at all.

Moreover, to be able to perform measurements of one or more rheological sizes continually, stationary appliances that are already familiar in the art are often too unwieldy or merely too large to be used comfortably by a patient over an extended period in a great range of activities. Devices that are usable entirely externally can, in addition, slide out of place on the human, animal, or plant organism during the usage phase, and thus in some cases can cause sensitive distortion in the measurements that are to be made. This can lead to repeated measurements that can be expensive. In fact, in a diagnosis that must be performed urgently, the time factor can often be critical.

### SUMMARY

Against this background, the present invention is based on the object of providing a mobile rheometric device for investigating rheological properties of human, animal, or plant components as well as a system and a method, which at least partly resolve the aforementioned disadvantages.

This object is fulfilled by means of a mobile rheometric device, a system, and a method having respective characteristics mentioned in the patent claims.

The invention accordingly foresees a mobile rheometric device for investigating rheological properties of human, animal, or plant components. Such a mobile rheometric device comprises a rheological measurement device and, coupled therewith, a computer-based functional unit with a user program. The mobile rheometric device here comprises a transport unit, in which the rheological measurement device and the computer-based functional unit coupled therewith are positioned. This transport unit, in the present context, is configured to be able to be placed at least temporarily in an internal component of a human, animal, or plant body, so that the rheological measurement device, upon coming into contact, can be interactive with the human, animal, or plant body.

In addition, a system for examining rheological properties of human, animal, or plant components is foreseen, which comprises at least one inventive mobile rheometric device and an external control device, with application program, that can be coupled contact-free with the at least one inventive mobile rheometric device. In addition, the system in this basic variant comprises at least one database that can be coupled with the control device for medical diagnosis of components of a human, animal, or plant body, wherein the database is based on rheological parameters.

In addition, a method for examining rheological properties of human, animal, or plant components is foreseen, said method comprising the following steps: providing and activation of an inventive mobile rheometric device, placement of the mobile rheometric device in or on a component of a human, animal, or plant body, triggering of at least one function of the rheological measurement device of the mobile rheometric device for interaction with at least one portion of the component of the human, animal, or plant body, so that at least one rheological parameter is measured and prepared by means of the rheological measurement device.

In addition, another use of the inventive mobile rheometric device is disclosed. The invention aims to make use of the inventive mobile rheometric device in performing therapeutic or healing measures in a human, animal, or plant organism.

An idea underlying the invention consists in providing a mobile rheometric device capable of conducting measurements of one or more rheological values, such as viscosities, sheer viscosities, extensional viscosities, sheer stresses, viscoelastic properties, storage module, loss module in or on a component of a human, animal, or plant body, and providing the measurement results for a more extensive diagnosis. Measurements completed by the rheological measurement device employed in the course of an interaction with the particular components of the human, animal, or plant body in this case can comprise absolute and also relative measurement values.

The proposed mobile rheometric device in this case is advantageously configured so that it can be inserted inside a component of the human, animal, or plant body. A transport unit designed for such purpose supports this method of insertion within the human, animal, or plant body that is to be examined. The components to be examined, in this context, can be understood as any subunits inside the human, animal, or plant body.

For example, in relation to a person, this can mean an organ unit of the body or generally a more complex structure within the human body, such as for instance a particular bone structure or the like.

The proposed inventive mobile rheometric device can also be inserted on the component of the human, animal, or plant body or organism that is to be examined. Use outside the respective body is also possible, for example in order to conduct possible calibration steps or the like before the actual insertion inside the body.

The advantage of the proposed mobile rheometric device is based, among other factors, on the fact that it can assure, in surprisingly simple means, that one or more rheological values can be determined continuously, while allowing comfortable operation thanks to its convenient functioning.

Moreover, because direct contact with the particular component that is to be examined is foreseen for purposes of measuring the aforementioned values, secure and methodical means of measurement can be assured. In addition, longer measurement periods can be expected by means of the inventive mobile rheometric device, because the application proceeds directly on the object that is to be investigated without additional restrictions for the organism being examined. For example, the mobile rheometric device can be placed in a human being in such a way that the patient is not restricted in their life activities to any notable way.

The proposed mobile rheometric device, furthermore, favors rapid procedures in communicating measurement results, since no further transports of previously extracted samples are required to reach an actual measurement device. In addition, the proposed mobile rheometric device makes it possible to dispense with cumbersome additional preparation of the component that is to be examined.

Interactions necessary for the rheological measurements can be completed rapidly and within limited time periods, virtually in real time, and thus a specific measurement or a series of measurements can be conducted in quick order and continuously.

Injury to areas of the sample that are to be measured can also be almost entirely ruled out, and thus a particularly safe method of taking measurements can be advantageously assured by means of the inventive device.

The inventive mobile rheometric device, in addition, can also be used advantageously in the course of treatment and diagnosis in remote regions, such as in mountainous areas for instance or at sea. The complex logistics and establishment of detailed infrastructure that are associated with large-scale apparatuses are no longer necessary, and thus the inventive mobile rheometric device, with its flexible and easy operation, offers a range of further advantages.

According to another embodiment variant of the mobile rheometric device, the transport unit comprises fastening means that are configured to assure temporary or longer-term stability of the mobile rheometric device in the internal component of the human, animal, or plant body.

In this manner it can be assured that even more time-consuming measurements, such as during recurring measurements at certain time intervals or the like, can essentially be performed comfortably and advantageously on the same portion of the component of the body that is to be examined.

After an initial placement, the mobile rheometric device can essentially remain at the said location so that the desired measurements can be completed in a comfortable and coherent way. At the same time, user-defined placement can be provided, offering significant protection, for example, for the organism that is to be examined.

A refinement of the mobile rheometric device foresees that the fastening methods are selected from the following:
inorganic fastenings; organic fastenings; inorganic fastenings designed to allow mechanical connection; organic fastenings designed to allow mechanical connection; inorganic fastenings designed to allow reversible mechanical connection; organic fastenings designed to allow reversible mechanical connection; inorganic fastenings designed to allow form-locking connection with at least a portion of the internal component; organic fastenings designed to allow form-locking connection with at least a portion of the internal component; inorganic fastenings designed to allow force-fitted connection with at least a portion of the internal component; organic fastenings designed to allow force-fitted connection with at least a portion of the internal component; inorganic fastenings designed to allow material bonding with at least a portion of the internal component; organic fastenings designed to allow material bonding with at least a portion of the internal component.

Thus, depending on the region and exact location, the use of a suited fastening can be selected, that is especially advantageous for the particular necessary purposes. Thus, for instance in a human body, an organic fastening can be considered particularly protective and also especially tolerable for the organism.

Another refinement of the mobile rheometric device foresees that the fastening should be at least partly mounted on a surface of the transport unit. In this manner, after placement of the device and upon contact with the particular component of the respective body and/or organism that is to be examined, fastening can immediately take place because, on the basis of the aforementioned special arrangement, a direct connection can be assured.

Another embodiment variant of the mobile rheometric device foresees that the fastening materials comprise fastenings designed to be capable of manipulation by means of at least one user-defined applied influence, so that the organic fastening means can be adapted to a molecular structure of the internal component of a human, animal, or plant body.

A particularly precise and thus especially stable connection can thus be obtained, so that the inventive device can be employed advantageously in a user-friendly and reliable way. In particular, and in accordance with the aforementioned advantages, a protective and thus highly tolerable technical solution for longer measurement periods can be provided by means of a specially adapted fastening, as described.

An additional embodiment variant of the mobile rheometric device foresees that the user-defined applied influence is selected from the following: thermal influence, electro-mechanical influence, mechanical influence, chemical influence, chemical, biological, or physical transmission of energy.

Depending on the intended purpose, therefore, an optimal fastening can be provided by means of the relevant technology. The particular influence to be adopted can be applied shortly before actual placement of the device.

A refinement of the mobile rheometric device foresees that the mobile rheometric device comprises at least one drive unit, so that the mobile rheometric unit is designed to be movable within the internal component of the human, animal, or plant body.

In this manner, in addition to an initial placement, the mobile rheometric device can be navigated with particular exactitude to a desired position in relation to the component that is to be examined. For purposes of fine adjustment, therefore, a particularly advantageous measurement site can be selected so that the subsequent measurements support the intended diagnosis or similar steps to the greatest extent possible.

In another embodiment of the mobile rheometric device, the at least one drive unit is configured to initiate a movement of the mobile rheometric device in a fluid in which the mobile rheometric device is situated, or whereby the at least one drive unit is configured to initiate a movement of the mobile rheometric device on an essentially firm foundation on which the mobile rheometric device is situated, or the at least one drive unit is configured to initiate a movement of the mobile rheometric device in a fluid in which the mobile rheometric device is situated and a movement of the mobile rheometric device on an essentially firm foundation on which the mobile rheometric device is situated.

For the various environmental conditions in the internal component of the human, animal, or plant body, therefore, a selected type and form of drive unit can be provided so that an optimal placement of the correspondingly configured mobile rheometric device can be achieved with exactitude at a desired site in relation to the component that is to be examined. Advantageous navigation can thus be more precisely assured in the sense of reaching a particularly advantageous measurement site.

An additional embodiment variant foresees that the at least one drive unit comprises at least one Taptic Engine unit.

The vibrations caused by means of the Taptic Engine can favor small-unit motion impulses of the mobile rheometric device, which can already be sufficient for a final placement. In particular, this procedure can be advantageous if only smaller displacement motions of the mobile rheometric device are desired, for example in order to hold a position already achieved. This can, moreover, be advantageous if external influences of the internal component of the human, animal, or plant body cause continually cause smaller displacements of the mobile rheometric device away from the optimal measurement site.

An alternative embodiment of the mobile rheometric device provides that the at least one drive unit is configured to be controlled by means of the computer-based functional unit.

Along with the possibility of performing various navigation corrections more precisely, there also exists the possibility of actuating additional, more distant measurement sites controllably. In other words, a controllable drive unit offers further advantages for eventually achieving an even better diagnosis, in that the greatest possible number of measurement values can be ascertained at different positions. An initial placement, which in some circumstances can be readjusted merely in a smaller area with a simple drive unit lacking a separate control, can also be reoriented by means of this embodiment variant in a user-defined manner in greater investigative areas, so that in each case measurements can be made at more distant measurement sites.

An additional embodiment foresees that the at least one drive unit consists at least partly of organic material.

The advantage here consists particularly in the fact that not only a tolerable implementation is hereby possible in the internal component of the human, animal, or plant body, but also that this tolerance is not worsened in the event of longer maneuvering.

A drive unit consisting at least partly of organic material is distinguished at times by not being immediately perceived as a foreign body, so that the probability of immediate negative reactions of the human, animal, or plant body can be considered slight.

Another embodiment variant foresees that the at least one drive unit comprises at least one movable outer membrane structure, which is disposed externally on at least one portion of the transport unit, so that a definite movement of the mobile rheometric device can be induced by means of pulsing motions of the particular movable outer membrane structure.

This innovation allows a particularly compact mobile rheometric device to be provided, which in addition does not comprise any protruding components that could be occasionally sensed as disturbing. Instead, such a movable outer membrane structure can adapt itself to an already present principal form of the mobile rheometric device, so that the aforementioned compact outer volume can be assured.

In an additional embodiment the at least one movable outer membrane structure can move by means of the computer-based functional unit with user program which is coupled for these purposes with the particular outer membrane structure, or wherein the at least one movable outer membrane structure is movable by means of an external stimulation unit with user program that is coupled in contact-free manner for these purposes with the respective outer membrane structure.

By means of a controllable movable outer membrane structure, the aforementioned advantages in both embodiments can be achieved even more effectively, both by means of the coupled computer-based functional unit with user program and also by means of the contact-less coupled external stimulation unit with user program.

A stimulation unit with user program can be equipped, for example, as any type of mobile control unit, such as a tablet or mobile telephone, each having a suitable user program, for instance in the form of an application or the like, so that this function is not necessarily required to be provided in the mobile rheometric device, which again results in a less complex and thus more user-friendly device.

In an additional embodiment variant of the mobile rheometric device, the at least one drive unit is configured to achieve a user-defined speed, in particular a user-defined consistent acceleration or a user-defined negative acceleration or a user-defined positive acceleration.

These various functions can either be pre-selected or else foreseen by means of a corresponding control unit. The related effects can each be used for a more controlled operation of the mobile rheometric device. Thus, for instance, a user-defined positive acceleration can favor a particularly stable fastening of the mobile rheometric device to this measurement site in the direction of a tissue area.

In another embodiment variant of the mobile rheometric device, the at least one drive unit is configured to induce any kind of movement or any kind of motion sequence of the mobile rheometric device in a three-dimensional coordinate system. The aforementioned advantages can be even more fully achieved as a result.

Another embodiment foresees that the computer-based functional unit comprises a storage unit for storing rheological measurements.

Thus, the ascertained measurements are not required to be transmitted immediately to additional devices, but instead can be staggered, or called up from the storage unit at a later time. This can be the case if the mobile rheometric device is removed from the corresponding body and thus free access to the storage unit is possible thereafter.

With extended testing periods, such as if a patient moves freely with the implemented mobile rheometric device, the acquired measurements can thus be stored by the storage unit without the need for a constant connection to reception devices outside.

In an additional embodiment variant of the mobile rheometric device, the computer-based functional element comprises a send-and-receive module, so that the mobile rheometric device is configured to transmit collected rheological measurements to at least one external device and in addition is configured to be operated by at least one external device.

This configuration offers the advantage that (after successful placement of the mobile rheometric device) the organism that is to be measured, whether it is now in human, animal, or plant form, is relatively free with respect to its location, since a connection to external devices, even over a certain distance, can be assured by means of the send-and-receive module. To this extent, virtually uninterrupted documentation by the collected measurements can be achieved even in real time, an advantage that in the case of critical values can signify a considerable time saving, so that curative measures or the like can be set in motion rapidly.

Another embodiment example provides that the mobile rheometric device comprises a tempering unit, so that the mobile rheometric device can maintain its temperature at least partially by user-defined means.

The advantage here is that subsequent rheological measurement processes, if useful for the respective method, can be optionally prearranged in that the mobile rheometric device-and thus its immediate surroundings at its measurement site in the body-can be tempered. This effect can also be obtained for therapeutic purposes. The tempering unit can thereby be configured to provide heat, cold, or both of those options selectively.

An additional embodiment provides that the mobile rheometric device comprises a temperature measurement unit, allowing measurement of any current temperature of at least a portion of the internal component of the human, animal, or plant body in which the mobile rheometric device is situated.

Thus, it is possible to insert the mobile rheometric device advantageously in order to track a particular temperature curve in the internal component of the human, animal, or plant body in which it has been placed and activated. In this manner, for example, inflammatory processes can be very precisely tracked, so that the collected measurements, in addition to the collected rheological measurements or parameters, can be put to use advantageously for a more precise analysis and/or an even more specific diagnosis.

In another embodiment of the invention, it is foreseen that the rheological measurement device is mounted adjustably by means of at least one movement device of the mobile rheometric device, so that it can be steered in the direction toward an area that is to be measured in the internal component of the human, animal, or plant body.

This embodiment can be of particular benefit if the first achieved measurement site is considered during the placement to be less than optimal. Thus, the measurement device can be reoriented by means of the at least one movement device, quickly and with a high degree of detail, so that the measurements that are subsequently to be obtained can be ascertained and/or measured with maximum efficiency.

According to an additional embodiment, the computer-based functional unit with user program is configured for classifying the measured rheological parameters of the human, animal, or plant body with respect to at least one medical diagnosis, so that a detailed report on the area of the human, animal, or plant body that is to be measured can be provided to a user of the mobile rheometric device.

This inventive variant of the mobile rheometric device offers the advantage that, thanks to the specially foreseen evaluation unit, classification of measurement values can be conducted directly on the spot. Thus, the mobile rheometric device not only takes measurements inside the respective body but also provides initial results because the classification supplies an initial estimation with respect to at least one medical diagnosis.

Another embodiment of the invention foresees that the computer-based functional unit and its user program can be combined with at least one database for medical diagnosis of human, animal, or plant bodies on the basis of rheological parameters, or at least comprises such a database.

In this manner a particularly rapid and comprehensive classification can be performed. Moreover, depending on the database used, the field of application of the inventive mobile rheometric device can be broadened. In turn, there is a greater likelihood, during application in distant regions, that the use of the inventive mobile rheometric device can be considered sufficient for a coherent analysis.

In this process, for instance, the rheological properties can be aligned with the database (or with several databases) providing information on the rheological properties of a physiological or pathological nature. In the course of this alignment, the rheometric device supports the description of a finding or of several findings for the diagnosis (or for the professional medical user) with respect to the condition of the sample. In addition, findings of a general nature in the form of parameters or the like can be deduced, in a form easily understandable and accessible for a medical layperson.

It thus becomes possible, by means of the foreseen mobile rheometric device and the corresponding method, to conduct rheological examinations with reference to a medical diagnosis of human, animal, or plant samples in the immediate vicinity and without delay. Such rheological examinations allow, for instance, measurements under the best physiological conditions possible, particularly with respect to a pH value, an ionic concentration, concerning the presence of enzymes or other parameters such as in body fluids.

In another embodiment the mobile rheometric device is configured to be placed in the interior of the human, animal, or plant body by means of catheterization technology. Not only does this method permit a very precisely aimed placement, since it is suitable for supporting accurate location efforts, but also it helps to assure that the mobile rheometric device can be implemented protectively and without risk of transport damage on approaching the initial site of measurement.

In the broadest sense of the medical-technical jargon, catheterization technology can be understood to mean any type of technology that allows a medical-technical unit, for example in the form of the inventive mobile rheometric device, to be transported into the interior of a body by means of a tube or hose apparatus or the like.

In this context the technical term signifies not only the actual placement of a tube device, hose device, or the like into the body but also the transport of the mobile rheometric device through this catheter as well as any type of auxiliary means and methodic steps serving placement of the mobile rheometric device initially in an internal component of the human, animal, or plant body.

An additional embodiment foresees that the mobile rheometric device is configured to be placed inside the human or animal body by oral insertion.

This can mean, for instance, that the foreseen mobile rheometric device in this particular embodiment should not exceed a certain outer volume that can allow oral insertion for a patient (human or animal). The advantage here is that placement can be achieved immediately and directly, without further techniques, without subjecting the patient to additional efforts.

In another embodiment, the dimensions of the mobile rheometric device are foreseen in the range between 2 m×2 m and 0.2 m×0.2 m, preferably between 1.5 m×2 m and 0.25 m×0.25 m, preferably between 1 m×1.5 m and 0.3 m×0.3 m, preferably between 1.5 m×1.5 m and 0.3 m×0.3 m, preferably between 0.5 m×0.5 m and 0.5 m×0.5 m.

Advantageously, therefore, a broad field of application is available to the mobile rheometric device. Transport to the place of application can also be considerably simplified, since the mobile rheometric device does not require especially large space even for fragile transport processes. For example, even transport via drone delivery technology and the like can be considered. Adjustable total weight of the device, for instance, can be estimated as between 45 kg and 250 kg, preferably between 45 kg and 150 kg, or even between 45 kg and 100 kg or between 45 kg and 70 kg.

### BRIEF DESCRIPTION OF THE DRAWINGS

The invention is described in greater detail hereinafter with reference to the drawings. The drawings are as follows:

FIG. 1 shows a schematic depiction of a mobile rheometric device according to one embodiment of the present invention;

FIG. 2 shows a schematic depiction of a user scenario of a mobile rheometric device according to one embodiment of the present invention;

FIG. 3 shows a schematic depiction of an additional user scenario of a mobile rheometric device according to an embodiment of the present invention;

FIG. 4 shows a schematic depiction of a system for examining rheological properties of human, animal, or plant components;

FIG. 5 shows a schematic flow diagram for a method according to one embodiment of the present invention.

Identical reference numbers in the drawings designate the same or functionally identical components, unless otherwise indicated.

### DETAILED DESCRIPTION

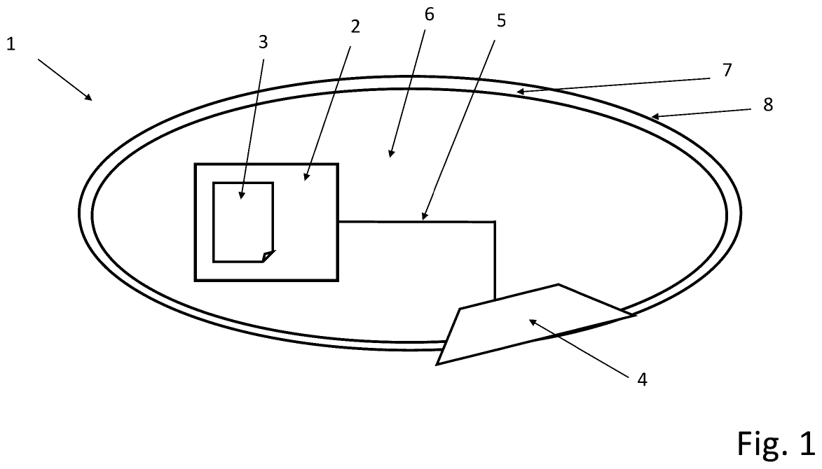

FIG. 1 shows a schematic depiction of a mobile rheometric device 1 according to one embodiment of the present invention. This mobile rheometric device 1 can be used and/or applied in investigating rheological properties of human, animal, or plant components.

For example, this can take the form of various internal components of a human, animal, or plant body. External application or use is not excluded here and can occur in the course of calibration processes before an actual interior use.

Examples of internal components of human or animal bodies can, for instance, be components of organs or even organs themselves. Thus the mobile rheometric device 1 can be employed in a stomach or in blood vessels, for instance. For example, the mobile rheometric device 1 can be configured for conducting examinations of bulking properties of a stomach lining.

For this purpose, the mobile rheometric device 1 can, for instance, be placed directly in or on a tissue of the stomach, such as the stomach lining. Likewise, a particular version of the mobile rheometric device 1 can be foreseen in order to make use of rheological properties at border areas such as at a phase boundary between a watery phase and an oily phase.

Likewise, a version of the mobile rheometric device 1 can be foreseen such as to perform measurements directly in a fluid volume, such as of a watery phase in the stomach contents, or to be used in connection with these purposes.

In other words, possible sites for the mobile rheometric device 1 in connection with a human or animal organism could be blood vessels or components of a digestive tract.

Components of a skin area can also be possible application sites. Similarly, other organs or components of the body such as the bladder or the like, can be possible sites for use of the mobile rheometric device 1.

The object of the rheological measurements that are to be taken here can be both the organs in themselves as well as any kind of bodily fluids or mixtures of bodily fluids and externally applied materials, such as a special food or generally any kind of nourishment components or even medical components or generally medicines or combinations of the aforementioned materials.

It is also possible to determine an application site in connection with an additional implant that is to be placed.

Conceivably as well, the mobile rheometric device 1 can be placed inside bones, so that for this purpose, for example, a cavity or the like must first be created in the bone and subsequently a placement must be made with or without supporting means in the said cavity or in the bones, in order to undertake or complete either a temporary placement or even a durable placement of the mobile rheometric device 1.

The mobile rheometric device 1 is depicted in FIG. 1 with its own computer-based functional element 2 with user program 3 and a rheological measurement device 4.

The rheological measurement device 4 here is shown coupled with the computer-based functional element 2 with user program 3, so that for these purposes a first connecting cable 5 is provided between these components.

In an embodiment variant (not shown in detail) it is conceivable that also more than one first connecting cable 5 is foreseen in order to assure a constantly secure connection in case a main connection should happen to be damaged or interrupted. It is also conceivable that any type of interfacing devices or at least partly radio-based connection technologies can be foreseen.

The rheological measurement device 4 and the computer-based functional unit 2 with user program 3, in addition, are depicted mounted in a transport unit 6 of the mobile rheometric device 1. In particular, both these components are shown positioned essentially in this transport unit 6 with respect to their respective external volume. Only a border area of the rheological measurement device 4 extends partly beyond an external border of this transport unit 6.

Conceivably, the rheological measurement device 4 is mounted in the transport unit 6 so that it can be adjusted by means of at least one movement device (not illustrated in detail) of the mobile rheometric device 1, and thus an external limiting of the rheological measurement device 4 in an initial operating condition can be foreseen positioned both inside the transport unit 6 and also, in an optional additional operating state, partly outside the transport unit 6.

These operating conditions can also vary or can be varied during a measurement and thus can be part of a measurement protocol contained in the user program 3.

The transport unit 6 here is configured to be placed, at least temporarily, inside an internal component (not illustrated further) of a human, animal, or plant body, so that the rheological measurement device 4 can interact with the human, animal, or plant body upon making contact.

Initiation of contact can be arranged in each case in such a way that a rheological measurement can be taken both of the components, for instance of tissue areas or the like, as well as of fluids, for example blood or stomach juices or other bodily fluids of human or animal organisms.

Analogously it is feasible to examine components of plants and any type of fluids of the plant to be measured in the course of the rheological measurements.

The actual rheological measurement proceeds, for example, with the rheological measuring device 4 itself causing a tensing or deformation, such as on the tissue or a bodily fluid.

Alternatively, the rheological measurement device 4, in an embodiment variant (not shown in detail), can be stimulated, extracorporeally or by an additional device, to vibrate or to perform a different motion. This can also be accomplished by means of at least one movement device (not illustrated in detail) of the mobile rheometric device 1. In this way, for instance, tissues can be examined, and thus pathological processes can be diagnosed and tracked over time.

In addition, such processes as, for instance, digestive processes can be examined. In this case the focus is not necessarily on the organ, the tissue, or a bodily fluid, but rather on the mixture of a chyme (or food pulp) with bodily fluid.

The effect of medications, for example in an administration (or dosing) during surgery, constitutes another possible use of the inventive mobile rheometric device 1.

The mobile rheometric device 1 can further be used to track a condition, for example of implants, and thus to determine at an early point just when an implant needs to be removed, for instance, or even replaced.

A major purpose for use of the inventive mobile rheometric device 1, in general, however, consists in obtaining rheological data or measurement values.

However, if it should be therapeutically beneficial from the medical viewpoint, the inventive mobile rheometric device 1 can also be used therapeutically, for example in selective stimulation of movements such as oscillation, or the application of heat/cold.

For such purposes, the inventive mobile rheometric device 1 (in an embodiment not illustrated in detail) can comprise at least one tempering unit.

In addition, the movably installed rheological measurement device 4 can be configured to transmit motion impulses to the organism that is to be examined, so that such motion impulses can be applied for therapeutic purposes. In this process, individual motion impulses or also motion impulse models or the like, can come into use.

The inventive mobile rheometric device 1, in addition, is depicted in FIG. 1 with a drive unit 7 in the form of a movable outer membrane structure 8.

This drive unit 7 in the form of a movable outer membrane structure 8 in this case is arranged in circular manner around the transport unit 6. To this extent it is disposed outside the transport 6, mounted on it.

Another embodiment (not illustrated in detail) is conceivably placed externally on at least one part of the transport unit 6. In any possible model, however, it is configured to generate a definite movement of the mobile rheometric device 1 by means of pulsing motions of the respective movable outer membrane structure 8.

Here the movable outer membrane structure 8 can be foreseen as coupled with the computer-based functional unit 2 with user program 3 for these purposes.

In other words, in such manner the mobile rheometric device 1 can be foreseen as directly movable by means of the computer-based functional unit 2 with user program 3 and as indirectly movable by means of the drive unit 7 in the form of the movable outer membrane structure. It can also be foreseen that the mobile rheometric device 1 can be movable both in fluids and on essentially firm foundations.

In another embodiment variant (not shown in detail), the drive unit 7 can also comprise additional forms. It can be, for example, a type of propeller device, which can be made essentially of organic-based material. Another alternative would be conventional mechanical units, which are predominantly made of inorganic material. Possible examples can be electrified drive units made of essentially inorganic materials.

The drive unit 7 in the form of a movable outer membrane structure 8, in an embodiment variant (not illustrated), can be designed as coupled with an external stimulation unit and user program, so that this stimulation unit with user program ultimately emits the impulses, so that the drive unit 7 in the form of a movable outer membrane structure 8 then moves the mobile rheometric device 1 such as from one initial measurement site to at least one additional measurement site. A user-defined navigation with any suitable auxiliary means can also be provided.

The transport unit 6, with the drive unit 7, in any variants can be designated together as a vehicle. Here it can be foreseen that the vehicle is optionally configured so that precise navigation is possible in the body to be examined. Accordingly, the vehicle, for instance on the basis of the drive unit 7, is especially configured for such purposes so that directed movements at a determined speed, or acceleration or surge or more frequent changes of location, are possible in the respective coordinate system.

In additional embodiment variants (not shown in detail), the mobile rheometric device 1, in addition, can comprise fastening devices (not illustrated) which are configured to support temporary or durable fastening of the mobile rheometric device 1 in the internal component of the human, animal, or plant body.

These fastening devices (not illustrated) can be selected from the following:
inorganic fastenings; organic fastenings; inorganic fastenings designed to allow mechanical connection; organic fastenings designed to allow mechanical connection; inorganic fastenings designed to allow reversible mechanical connection; organic fastenings designed to allow reversible mechanical connection; inorganic fastenings designed to allow form-locking connection with at least a portion of the internal component; organic fastenings designed to allow form-locking connection with at least a portion of the internal component; inorganic fastenings designed to allow force-fitted connection with at least a portion of the internal component; organic fastenings designed to allow force-fitted connection with at least a portion of the internal component; inorganic fastenings designed to allow material bonding with at least a portion of the internal component; organic fastenings designed to allow material bonding with at least a portion of the internal component.

For example, any of the aforementioned organic fastening devices can be based on biological fastening processes and are designed for such purposes.

To this extent it is conceivable that the organic fastening devices are configured to forge various connections by means of biological bridging with a tissue or other biological component of the body to be examined with the ultimate aim of ensuring that the mobile rheometric device 1 is essentially fixed in place, for instance that it can be attached or fastened onto an initial measurement point in the body to be examined.

Use is made here of the fact that human cells comprise a certain spatial characteristic, causing three-dimensional protrusion, for instance, into a spatial volume so that a connection is possible here, for instance by means of special protein structures of the organic fastening devices.

This can be visually understood in the sense of a principle of two puzzle pieces. Generally, moreover, such organic connections can be provided by means of corresponding molecular bridges, so that for instance a key-and-lock model or the like can be imagined. Here, various loadings are possible on the partners to be connected, so that at least a corresponding molecular bridge can be foreseen on the basis of loading differences.

The inventive mobile rheometric device 1 can be placed in or on the organ that is to be examined in or on the human, animal, or plant body, for instance by catheterization or the like or by oral intake or manual insertion. In addition, surgical placement of the inventive mobile rheometric device 1 can be foreseen. This can be achieved by minimally invasive methods.

For example, a hole can be bored into a bone and then the mobile rheometric device 1 is placed in the hole and finally use is made of a kind of organic cement or a tolerable gluing material or other supporting means for securing the mobile rheometric device 1 in the hole.

For the evaluation, access can optionally be made to a database system, wherein such a database system for instance can be made available globally or locally on a (mobile) end-user device, a cloud device, or else on the network of a practice (or joint practice) or clinic.

On the basis of this database system, it becomes possible to provide a diagnosis or the description of trends of physiological or pathological conditions associated with certain (suspected) diagnoses.

Optionally a data transmission onto a mobile end-user device can occur, for example when a patient is tracking the course of their own illness as a layperson.

For purposes of evaluation, measured data and/or measurement values can, in one option, be stored in a storage unit (not illustrated) of the mobile rheometric device 1 (for instance, for the duration of a particular period of time) and then can be evaluated.

Alternatively, communication can take place by means of a send-and-receive module (not illustrated) of the mobile rheometric device 1 in the form of data transmission. This can occur as early as during the period in which the mobile rheometric device 1 is still situated inside the body to be measured.

To this extent, data transmission in real time is also conceivable with corresponding visualization on an imaging device, a monitor unit, or the like.

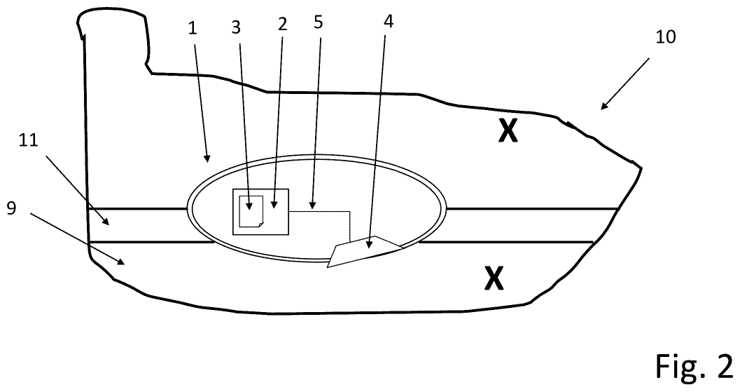

FIG. 2 shows a schematic depiction of a user scenario of a mobile rheometric device 1 according to an embodiment of the present invention. This can take the form of particular embodiment variants that have been mentioned in the description of FIG. 1.

Identical reference numerals designate the same characteristics, which thus need not be repeated here.

The mobile rheometric device 1 illustrated in FIG. 2 is submerged here in a watery phase 9 in a human stomach 10, in such a way that it also floats to some extent in an oily phase 11 situated upon the watery phase 9.

The two X symbols represent possible additional sites at which the mobile rheometric device 1 can be secured inside the stomach 10 by means of fastenings (not illustrated).

The lower X symbol (on the basis of the image plane) shows that the mobile rheometric device 1 can also be entirely situated in the watery phase 9 in the stomach 10. In corresponding manner, the upper X symbol indicates (again on the basis of the image plane) that the mobile rheometric device 1 can also be situated entirely outside both the watery phase 9 and the oily phase 11.

The said device can be situated there in order to examine bulking properties of a portion of the lining of the stomach 10. Correspondingly, bulking properties can be investigated in the water phase 9 in this area of the lining of the stomach 10.

Conceivably, in certain embodiments (not illustrated) the mobile rheometric device 1 can counteract such displacements of location by means of the drive unit 7 in the form of a movable outer membrane structure 8, while releasable fastening devices can be advantageous so that, after a change of location, a stable securing of location can be made without thereby endangering structures of the mobile rheometric device 1.

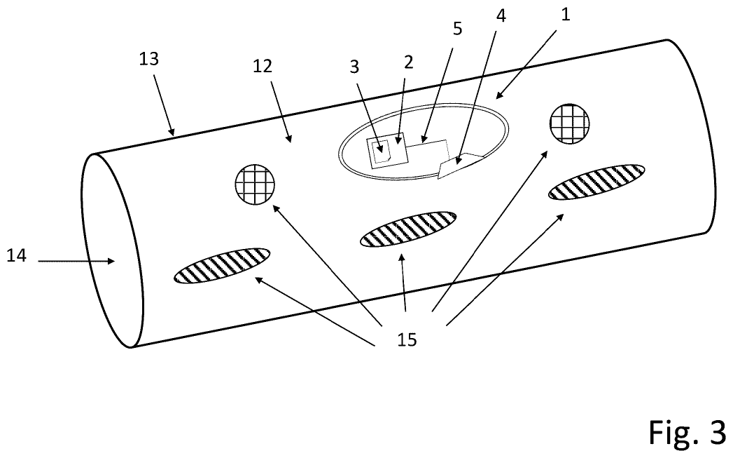

FIG. 3 shows a schematic depiction of an additional user scenario of a mobile rheometric device 1 according to an embodiment of the present invention.

This can involve, for instance, various embodiment variants which have been mentioned in the description of FIG. 1. Identical reference numerals designate identical characteristics, which thus need not be repeated here.

The mobile rheometric device 1 in FIG. 3 is shown in especially small format, wherein it can comprise for instance a maximum outer volume of no more than one-half of the diameter of a fairly large blood vessel. To this extent a maximum outer volume of up to 500 micrometers is foreseeable.

The mobile rheometric device 1 in FIG. 3 is schematically depicted as mounted on an inner wall 12 of a blood vessel 13, for example a large human artery.

This can be achieved, for example, by means of inorganic or organic fastening devices (not illustrated).

A sensor surface (not illustrated, and/or not visible) of the rheological measurement device 4 here is directed toward an interior space 14 of the blood vessel 13, so that rheological measurement is possible on the fluid as it flows past, that is, on human blood with its components (schematically very simplified and not true to scale in the illustration by means of its blood components 15).

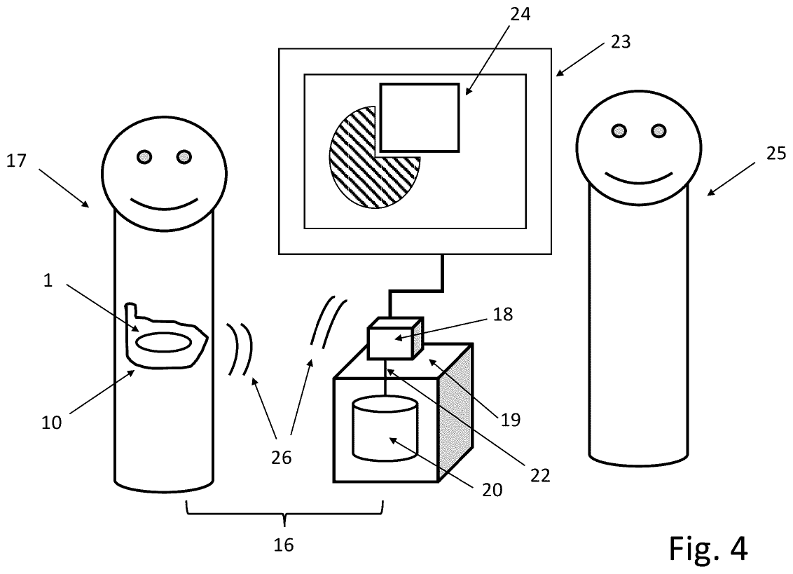

FIG. 4 shows a schematic depiction of a system 16 for examining rheological properties of human, animal, or plant components.

A mobile rheometric device 1 according to an embodiment of the present invention is depicted here in a stomach 10 of a patient 17.

In one embodiment variant (not illustrated) more than one mobile rheometric device 1 can be foreseen within the body of the patient 17.

The illustrated system 16 here is configured for examining rheological properties of human, animal, or plant components and for this purpose comprises, in addition to the inventive mobile rheometric device 1, an external control device 18 and application program, which is shown on a depot structure 19 alongside the patient 17 and can be coupled contact-free with the at least one mobile rheometric device 1.

The depot structure 19 can be a table or some other piece of furniture of the kind used in a hospital, particularly in an operating room.

Below the external control device 18 (on the basis of the image plane), a database 20 is shown in very simplified, merely symbolic form in the depot structure 19.

This database 20 is shown as coupled with the external control device 18 by a second connecting cable 21. The database 20 in this case is a computer-technical device for medical diagnosis of components of a human, animal, or plant body on the basis of rheological parameters.

It can be foreseen, in a variant (not illustrated), that more than one database 20 is coupled with the external control device 18. For example, there can be various databases from a group medical practice, a cloud device, a hospital network, a research network, and the like.

The external control device 18 is shown coupled with a display unit 23 by a third connecting cable 22. In very simplified form, a diagnostic image 24 of the rheological examination is shown on the display unit 23, which in one embodiment can optionally be an active component of the system 16.

The display unit 23 can also be foreseen as a separate component and the system 16 as having merely one external control unit 18 available, which is equipped with the standard interfacing devices for coupling of additional external devices.

The display unit 23, in each of the foregoing cases, can be a type of flat-screen unit or the like. A medical specialist 25 is shown standing (on the basis of the image plane) to the right of the display unit 23, reviewing the diagnostic image 24 currently displayed.

Illustrated radio wave symbols 26 depict the communication process of the mobile rheometric device 1 inside the body of the patient 17 with the external control device 18.

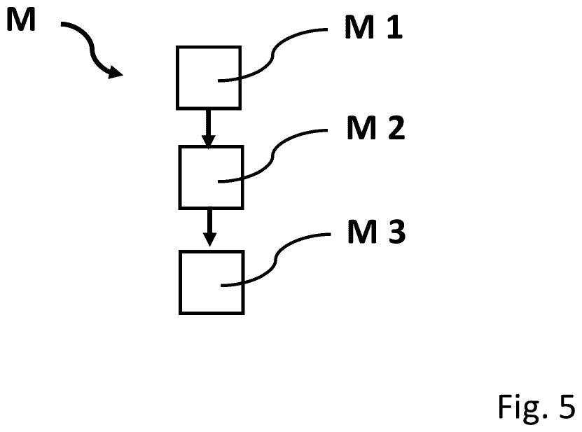

FIG. 5 shows a schematic flow diagram for a method M for examining rheological properties of human, animal, or plant components. In a first step M1 of the method, an inventive mobile rheometric device 1 is provided and activated. In a second step M2 of the method, an inventive mobile rheometric device 1 is placed in or on a component of a human, animal, or plant body. In a third step M3 of the method, at least one function of the rheological measurement device 4 of the mobile rheometric device 1 is triggered for interaction with at least one portion of the component of the human, animal, or plant body, so that at least one rheological parameter is measured and provided by means of the rheological measurement device.

1. A mobile rheometric device for investigating rheological properties of human, animal, or plant components, comprising a rheological measurement device and, coupled thereto, a computer-based functional unit with user program, wherein the mobile rheometric device includes a transport unit in which the rheological measurement device and the computer-based functional unit coupled thereto are positioned, wherein the transport unit is configured to be placed at least temporarily in an internal component of a human, animal, or plant body so that the rheological measurement device can interact with the human, animal, or plant body upon contact.

2. The mobile rheometric device according to claim 1, wherein the transport unit comprises fastening devices, which are configured to make temporary or durable fastening of the mobile rheometric device possible in the internal component of the human, animal, or plant body.

3. The mobile rheometric device according to claim 2, wherein the fastening device is selected from the following:
inorganic fastenings; organic fastenings; inorganic fastenings designed to allow mechanical connection; organic fastenings designed to allow mechanical connection; inorganic fastenings designed to allow reversible mechanical connection; organic fastenings designed to allow reversible mechanical connection; inorganic fastenings designed to allow form-locking connection with at least a portion of the internal component; organic fastenings designed to allow form-locking connection with at least a portion of the internal component; inorganic fastenings designed to allow force-fitted connection with at least a portion of the internal component; organic fastenings designed to allow force-fitted connection with at least a portion of the internal component; inorganic fastenings designed to allow material bonding with at least a portion of the internal component; organic fastenings designed to allow material bonding with at least a portion of the internal component, and combinations of any of these.

4. The mobile rheometric device according to claim 2, wherein the fastening devices are at least partly mounted on a surface of the transport unit

5. The mobile rheometric device according to claim 2, wherein the fastening devices comprise organic fastening means configured to be capable of manipulation by means of at least one user-defined applied influence, so that the organic fastening means can be adjusted to a molecular structure of the internal component of a human, animal, or plant body.

6. The mobile rheometric device according to claim 5, wherein the user-defined applied influence is selected from the following: thermal influence: electro-mechanical influence; mechanical influence; chemical influence; chemical, biological, or physical transmission of energy; and combinations of any of these.

7. The mobile rheometric device according to claim 1, wherein the mobile rheometric device comprises at least one drive unit, so that the mobile rheometric device can be configured to be movable inside the internal component of the human, animal, or plant body.

8. The mobile rheometric device according to claim 7, wherein the at least one drive unit is configured to induce a movement of the mobile rheometric device in a fluid in which the mobile rheometric device is situated or wherein the at least one drive unit is configured to induce a movement of the mobile rheometric device on an essentially firm foundation on which the mobile rheometric device is situated, or wherein the at least one drive unit is configured to induce a movement of the mobile rheometric device in a fluid in which the mobile rheometric device is situated and a movement of the mobile rheometric device on an essentially firm foundation on which the mobile rheometric device is situated.

9. The mobile rheometric device according to claim 7, wherein the at least one drive unit comprises at least one Taptic Engine unit.

10. The mobile rheometric device according to claim 7, wherein the at least one drive unit is configured to be controlled by means of the computer-based functional unit.

11. The mobile rheometric device according to claim 7, wherein the at least one drive unit is provided at least partly of organic material.

12. The mobile rheometric device according to claim 7, wherein the at least one drive unit comprises at least one movable outer membrane structure, which is positioned outside on at least one portion of the transport unit, so that a defined motion of the mobile rheometric device can be caused by means of pulsing motions of the particular movable outer membrane structure.

13. The mobile rheometric device according to claim 12, wherein the at least one movable outer membrane structure can be moved by means of the computer-based functional unit with user program coupled for these purposes with the particular outer membrane structure or wherein the at least one movable outer membrane structure can be moved by means of an external stimulation unit with user program that is coupled contact-free for these purposes with the particular outer membrane structure.

14. The mobile rheometric device according to claim 7, wherein the at least one drive unit is configured to induce a user-defined speed, in particular a user-defined uniform acceleration or a user-defined negative acceleration or a user-defined positive acceleration.

15. The mobile rheometric device according to claim 1, wherein the at least one drive unit is configured to induce any kind of movement or any kind of motion sequence of the mobile rheometric device in a three-dimensional coordinate system.

16. The mobile rheometric device according to claim 1, wherein the computer-based functional unit comprises a storage unit for storing rheological measurement data.

17. The mobile rheometric device according to claim 1, wherein the computer-based functional unit comprises a send-and-receive module, so that the mobile rheometric device is configured to transmit collected rheological measurement data to at least one external device and in addition is configured to be operated by at least one external device.

18. The mobile rheometric device according to claim 1, wherein the mobile rheometric device comprises a tempering unit so that the mobile rheometric device can be brought to the intended temperature, at least partly by user-defined input.

19. The mobile rheometric device according to claim 1, wherein the mobile rheometric device comprises a temperature measurement unit so that any current temperature of at least one portion of the internal component of the human, animal, or plant body in which the mobile rheometric device is situated can be measured.

20. The mobile rheometric device according to claim 1, wherein the rheological measurement device can be mounted adjustably by means of at least one movement device of the mobile rheometric device, so that it can be adjustably guided in the direction of an area that is to be measured in the internal component of the human, animal, or plant body.

21. The mobile rheometric device according to claim 1, wherein the computer-based functional unit with user program is configured to classify the measured rheological parameters of the human, animal, or plant body with respect to at least one medical diagnosis, so that a detailed report on the area of the human, animal, or plant body that is to be measured can be provided to a user of the mobile rheometric device.

22. The mobile rheometric device according to claim 21, wherein the computer-based functional unit with user program can be connected with at least one database for medical diagnosis of human, animal, or plant bodies on the basis of rheological parameters, or at least includes such a database.

23. The mobile rheometric device according to claim 1, wherein the mobile rheometric device is configured to be placed inside the human, animal, or plant body by means of catheterizing technology.

24. The mobile rheometric device according to claim 1, wherein the mobile rheometric device is configured to be inserted into the human or animal body by oral intake.

25. The mobile rheometric device according to claim 1, wherein the outer dimensions of the mobile rheometric device are foreseen as being in the range between 2 m×2 m and 0.2 m×0.2 m, preferably between 1.5 m×2 m and 0.25 m×0.25 m, preferably between 1 m×1.5 m and 0.3 m×0.3 m, preferably between 1.5 m×1.5 and 0.3 m×0.3 m, preferably between 0.5 m×0.5 m and 0.5 m×0.5 m.

26. A system for supporting rheological properties of human, animal, or plant components comprising at least one mobile rheometric device for investigating rheological properties of human, animal, or plant components, including a rheological measurement device and, coupled thereto, a computer-based functional unit with user program, wherein the mobile rheometric device includes a transport unit in which the rheological measurement device and the computer-based functional unit coupled thereto are positioned, wherein the transport unit is configured to be placed at least temporarily in an internal component of a human, animal, or plant body so that the rheological measurement device can interact with the human, animal, or plant body upon contact, an external control device with application program that can be coupled contact-free with the at least one mobile rheometric device, and at least one database that can be coupled with the control device for medical diagnosis of components of a human, animal, or plant body on the basis of rheological parameters.

27. A method for examining rheological properties of human, animal, or plant components, comprising:
providing and activating a mobile rheometric device for investigating rheological properties of human, animal, or plant components, including a rheological measurement device and, coupled thereto, a computer-based functional unit with user program, wherein the mobile rheometric device includes a transport unit in which the rheological measurement device and the computer-based functional unit coupled thereto are positioned, wherein the transport unit is configured to be placed at least temporarily in an internal component of a human, animal, or plant body so that the rheological measurement device can interact with the human, animal, or plant body upon contact;
placement of the mobile rheometric device in or on a component of a human, animal, or plant body; and
triggering of at least one function of the rheological measurement device of the mobile rheometric device for interaction with at least one portion of the component of the human, animal, or plant body, so that at least one rheological parameter is measured and provided by means of the rheological measurement unit.

28. A use of a mobile rheometric device for investigating rheological properties of human, animal, or plant components, including a rheological measurement device and, coupled thereto, a computer-based functional unit with user program, wherein the mobile rheometric device includes a transport unit in which the rheological measurement device and the computer-based functional unit coupled thereto are positioned, wherein the transport unit is configured to be placed at least temporarily in an internal component of a human, animal, or plant body so that the rheological measurement device can interact with the human, animal, or plant body upon contact, and for performing therapeutic or healing measures in a human, animal, or plant organism.
